(19) United States
(12) Patent Application Publication
(10) Pub. No.: US 2022/0042962 A1
(43) Pub. Date: Feb. 10, 2022

(54) METHOD FOR VALIDATION OF SITE-SPECIFIC WATER QUALITY CRITERIA OF RIVER BASIN

(71) Applicant: DALIAN UNIVERSITY OF TECHNOLOGY, Dalian, Liaoning (CN)

(72) Inventors: Zhihong LIU, Dalian, Liaoning (CN); Zhuohang XIN, Dalian, Liaoning (CN); Chi ZHANG, Dalian, Liaoning (CN); Changchun SONG, Dalian, Liaoning (CN)

(21) Appl. No.: 17/258,401

(22) PCT Filed: Feb. 28, 2020
(86) PCT No.: PCT/CN2020/077093

§ 371 Date: Jan. 6, 2021

(30) Foreign Application Priority Data
Jan. 9, 2020 (CN) 202010020553.9

(51) Int. Cl. G01N 33/18

(52) U.S. Cl. CPC G01N 33/18

(57) ABSTRACT

A method for validation of site-specific water quality criteria of a river basin includes steps of: analyzing the biota distribution characteristics of a river basin; eliminating non-resident related species from the toxicity database of target pollutants; supplementing toxicity values of site-specific resident sensitive species and endemic species; establishing a resident toxicity database for the river basin; comparing the degrees of fitting of species sensitivity distribution (SSD) models and identifying the optimal SSD model; and validating the water quality criterion value to complete the derivation and validation of the site-specific water quality criteria of the river basin. The method can improve the nativeness of the site-specific water quality criteria of the river basin, be used for the formulation of the site-specific water environment quality standards of the river basin, and provide support for the water quality management of the river basin.

### TECHNICAL FIELD

The present invention belongs to the field of derivation and validation of water quality criteria, and relates to a method for validation of site-specific water quality criteria of a river basin.

### BACKGROUND

Water quality criteria provide a scientific basis for formulating water quality standards and are one of the important methods of water quality management. The water quality criteria are usually based on the aquatic toxicity values of related species in a region, and derived by using a model. The nativeness of biological toxicity values has an important impact on the representativeness and applicability of water quality criteria.

How to determine the resident biological toxicity database that fits the site-specific fauna distribution characteristics of the river basin is a more difficult problem in derivation and validation of water quality criteria at the river basin level than at the national level, and the loss of resident toxicity values, especially the loss of resident toxicity values, is the main bottleneck in the derivation of the water quality criteria of the river basin and the main reason for the large uncertainty of the site-specific water quality criterion values. The scientificity and accuracy of the water quality criteria depend on whether the toxicity values are natively representative. At present, the relatively mature methods are provided at home and abroad for derivation of the water quality criteria at the national level to identify the test species so as to establish the biological toxicity database and derive the national water quality criteria. However, many problems still exist in validation of the site-specific water quality criteria at the river basin level, and the core of the problems is: (1) how to solve the problem of loss of toxicity values of resident species and (2) how to emphasize the toxicity values of site-specific sensitive species and endemic species in the toxicity database. At present, the screening of the resident species lacks instructional systems and methods in the derivation of the water quality criteria of the river basin, and meanwhile, the emphasis of the toxicity values of resident species is not reflected in the process of derivation. As a result, the derivation result of the water quality criteria has large uncertainty and thus is difficult to guide the water environment management of the river basin.

The scientific management of water bodies cannot be performed without the scientific formulation of the water quality criteria. This importance is reflected not only at the national level, but also at the specific river basin level. The needs of the site-specific water quality management of the river basin can be met only by improving the nativeness and applicability of the site-specific water quality criteria. Therefore, the development of the water quality criteria that can reflect site-specific characteristics is of great significance to the protection and scientific management of water bodies in the river basin.

### SUMMARY

In view of the problems in the prior art, the present invention provides a method for validation of site-specific water quality criteria of a river basin, which provides support for the water environment protection and the development of the site-specific water quality criteria at the specific river basin level.

To achieve the above purpose, the present invention adopts the following technical solution:

A method for validation of site-specific water quality criteria of a river basin, comprises the following steps:

Step 1: analyzing the biota distribution characteristics of a river basin.

1.1) Collecting the zoography and local literature yearbooks of the river basin and summarizing species categories in the river basin;

1.2) Sorting all site-specific species in order of genus, family and order from low to high according to biological taxonomy levels;

1.3) Marking the endemic species according to the biological distribution characteristics of the river basin in combination with the data query of the species distribution area.

1.4) Summarizing the biota distribution characteristics of the river basin, including the number of species, the proportion of species corresponding to different biological taxonomy levels (such as genus, family and order) and the species and taxon distribution of endemic species.

Step 2, eliminating non-resident related species from the existing toxicity values.

2.1) Collecting, screening and summarizing the aquatic toxicity values of target pollutants;

2.2) Comparing the resident species with the biological species having toxicity values in the river basin;

2.3) Marking the species completely corresponding to the species names;

2.4) Marking and reserving the species with different species names and the same species (genus and family) level based on biological classification as the reference;

2.5) Eliminating other species that do not meet the requirements in steps 2.3) and 2.4).

Step 3, supplementing toxicity values of site-specific resident sensitive species and endemic species.

3.1) Screening the toxicity values of the species, wherein the screening principle is: the toxicity test subjects and the test process required to obtain toxicity values meet the requirements of the relevant toxicity test specifications, and after all qualified toxicity values of the species are screened, the calculation formula of the species mean acute values (SMAV) of the finally reserved species in step 2 is as follows:

$$S M A V = \sqrt[n]{E {C}_{501} \times E {C}_{502} \times E {C}_{503} \times … \times {EC}_{50 n}}$$

wherein EC₅₀₁-EC₅₀ₙ are the toxicity values of the same species, and n is the number of toxicity values of the same species; and EC50 (median effect concentration) can be substituted by LC50 (median lethal concentration).

After obtaining the SMAVs of all species, sorting the SMAVs from small to large;

3.2) Selecting four species with the minimum SMAVs, and identifying four sensitive test species corresponding to four families according to the biological taxonomy of the species; in principle, each family corresponds to one species, but if no available test species exists in a family (if the species of the family belongs to protected animals, or the experimental availability is not strong), amplifying one species from the higher sensitive family or identifying one substitute species from the families under sorting;

3.3) Based on the family corresponding to the species with the minimum SMAV, identifying two endemic test species among the endemic species in the river basin;

3.4) Conducting toxicity tests on the sensitive test species identified in step 3.2) and the endemic test species identified in step 3.3), and setting control tests for the toxicity tests, wherein the test species, exposure conditions and test procedures of the control group and the experimental group shall be exactly the same, the exposure concentration shall be set based on the principle of equal ratio, the SPSS linear regression method is used to calculate EC50 or LC50 as the supplementary toxicity value, and the ratio in the principle of equal ratio is 2.

Step 4, establishing a resident toxicity database for the river basin.

4.1) If the sensitive test species already has a toxicity value, using the newly obtained toxicity value to replace the original toxicity value of the species;

4.2) Adding the sensitive test species without previous toxicity values and the toxicity values of endemic test species to the original toxicity database;

4.3) According to the toxicity values, sorting all the species in ascending order to form the resident toxicity database of the river basin.

Step 5, comparing the degrees of fitting of species sensitivity distribution (SSD) models and identifying the optimal SSD model.

5.1) Calculating the cumulative probability P of the species, wherein the calculation method is as follows: the species with the minimum toxicity value is assigned a value of 1, by analogy, the species with the maximum toxicity value is assigned a value of n a total of n species are assumed, and the cumulative probability of the species is P=r/n+1, wherein n is the number of the species in sorting;

5.2) Taking all base-10 natural logarithmic values as toxicity values;

5.3) With the logarithm values of the toxicity values as independent variables and the cumulative probabilities of the species as dependent variables, respectively using normal, logistic and BurIII distribution models for fitting (using Origin for fitting) to obtain three different fit coefficients R²; and using the maximum fit coefficient R2 as the final fitting model for derivation of criteria as the optimum SSD model.

The functional relations of the normal distribution model, the logistic distribution model and the BurIII distribution model are as follows:

The functional relation of the normal model is y=(1/(a*(2π)\hat{ }0.5))*exp((−(x−b)\hat{ }2)/(2*a\hat{ }2));

wherein y is a dependent variable, a is one of model parameters, x is an independent variable, and b is one of model parameters.

The functional relation of the logistic model is y=1/exp(−(x−a)/b);

wherein y is a dependent variable, a is one of model parameters, x is an independent variable, and b is one of model parameters.

The functional relation of the BurrIII model is y=1/(1+(1+a/x)\hat{ }b)\hat{ }c;

wherein y is a dependent variable, a is one of model parameters, x is an independent variable, b is one of model parameters, and c is one of model parameters.

Step 6, validating the water quality criterion value.

6.1) With the logarithm value of the toxicity value in the resident toxicity database of the river basin determined in step 4 as the X variable and the cumulative probability of the species as the Y variable, adopting the fitting models identified in step 5.3) for fitting;

6.2) Taking the X value corresponding to Y=0.05 for base-10 exponent transformation and then dividing by the safety coefficient M to obtain that the site-specific water quality criterion value of the river basin of the target pollutant is 10^{X}/M (the criterion is obtained by the species sensitivity method), wherein the safety coefficient R is 2.

6.3) Based on the toxicity percentage sorting method, calculating the final toxicity value (FV) by using the toxicity values of four most sensitive species among the resident species and the sensitive species, wherein the calculation formula is as follows:

$${S}^{2} = \frac{\sum [ {( ln SMAV )}^{2} ] - [ {( \sum ln SMAV )}^{2} / 4 ]}{\sum ( P ) - \frac{{[ \sum ( \sqrt{P} ) ]}^{2}}{4}} \\ L = \{ \sum ( ln SMAV ) - S [ \sum ( \sqrt{P} ) ] \} / 4 \\ A = S ( \sqrt{0.05} ) + L \\ F V = {e}^{A} \qquad$$

wherein S, L and A are respectively the parameters generated in the calculation process, SMAV is the species mean acute value, P is the cumulative probability corresponding to the species, and FV is the final toxicity value.

Dividing FV obtained by derivation by the safety coefficient 2 to obtain the site-specific water quality criterion of the river basin (which is obtained by the toxicity sorting method)

6.4) According to the resident sensitive species and the sorting of the toxicity of the sensitive species in the site-specific resident toxicity database, respectively selecting the weight values obtained by two different derivation methods (species sensitivity method and toxicity sorting method);
Table 1 shows the weight values corresponding to different derivation methods based on the ranges of the average cumulative probabilities of the resident sensitive species and the endemic species.

| | Range of Average Cumulative | | | |
|---|---|---|---|---|
| | Probabilities of Resident Sensitive | | | |
| | Species and Endemic Species | | | |
| | 0-0.30 | 0.31-0.50 | 0.51-0.80 | 0.81-1.0 |
| Weight value obtained based | 0.3 | 0.5 | 0.8 | 1.0 |
| on species sensitivity method | | | | |
| Weight value obtained based | 0.7 | 0.5 | 0.2 | 0.0 |
| on toxicity sorting method | | | | |

According to the weight values in the table above, obtaining the site-specific water quality criterion value of the river basin, as shown in the following formula:

WQC=WQCs×a+WQCr×b

wherein WQC is the final site-specific water quality criterion value of the river basin, WQCs is the site-specific water quality criterion value of the river basin derived by the species sensitivity method, WQCr is the site-specific water quality criterion value of the river basin derived by the toxicity sorting method, a is the weight obtained based on the average cumulative probability of the resident sensitive species, and b is the weight obtained based on the average cumulative probability of the endemic species;

The present invention has the following beneficial effects: the resident sensitive species and the endemic species can be screened accurately, the toxicity values of resident species are emphatically reflected in the process of criterion validation, and the nativeness and applicability of the site-specific water quality criteria of the river basin are greatly improved so as to better serve the water environment management of the river basin.

### DESCRIPTION OF DRAWINGS

FIG. 1 is a flow chart of validation of site-specific water quality criteria of a river basin of the present invention.

FIG. 2 shows a sensitivity distribution curve of aquatic species in Liaohe River Basin in embodiments of the present invention.

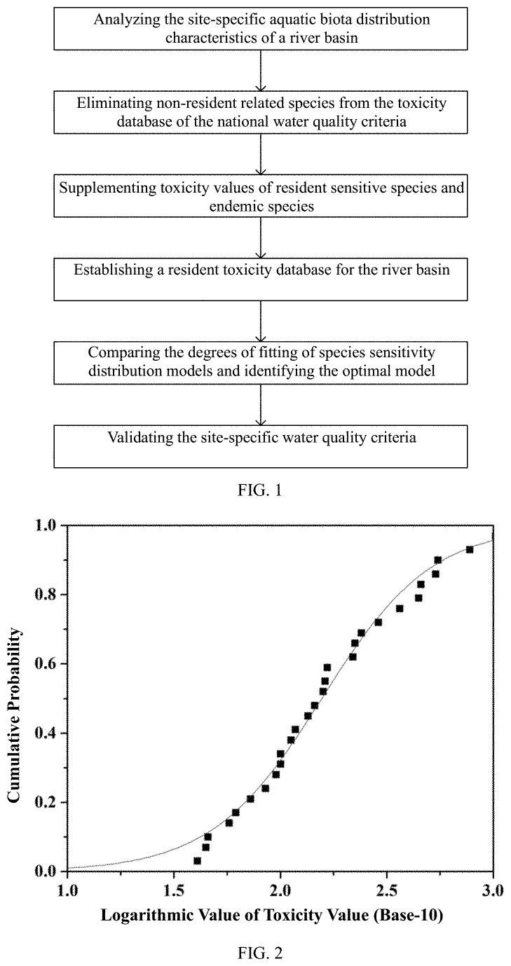

### DETAILED DESCRIPTION

The present invention relates to a method for validation of site-specific water quality criteria of a river basin, which is suitable for validation of site-specific water quality criteria of river basins that have water quality indexes of the national water quality criteria.

The present invention will be further described in detail below in combination with specific embodiments.

The validation process of ammonia nitrogen water quality criteria of Liaohe River Basin in China is as follows.

Step 1: analyzing the biota distribution characteristics of a river basin; Collecting the databases of Fauna Sinica, Liaoning Fauna, and China National Knowledge Infrastructure (CNKI), summarizing the distribution information of aquatic species in Liaohe River Basin, and classifying and summarizing the collected distribution information of aquatic species in Liaohe River Basin in order of species, genus and family based on biological classification. According to the available data, in terms of aquatic species, about 96 species of vertebrates and 291 species of invertebrates are distributed in Liaohe River Basin.

Step 2, eliminating non-resident related species from the existing toxicity values; Collecting aquatic toxicity values with ammonia nitrogen as the target pollutant, including toxicity database (ECOTOX) and ammonia nitrogen water quality criterion derivation guideline of the United States Environmental Protection Agency (USEPA), database of China National Knowledge Infrastructure (CNKI), Google Scholar and other data sources. Ammonia nitrogen aquatic toxicity values covering about 120 species are collected; after eliminating species by comparing the aquatic biota distribution characteristics of Liaohe River Basin, a total of 25 aquatic species with native correlation with Liaohe River Basin are screened, wherein 16 species are distributed in Liaohe River Basin and the other 9 species are substitute species, i.e., the species are not distributed in Liaohe River Basin, but the species of the same family as the corresponding species are distributed in Liaohe River Basin. See Table 1.

TABLE 1
Toxicity Database of Existing Species in Liaohe River Basin (all toxicity
values are based on the conditions of pH = 7.0 and temperature = 20° C.)

| | | | Species in | | Species Mean |
|---|---|---|---|---|---|
| | | | Liaohe River | Substitute | Acute Value |
| Order | Species | Family | Basin | Species* | (mg/L) |
| 1 | Leuciscus cephalus | Cyprinidae | | Yes | 40.9 |
| 2 | Acipenser sinensis | Acipenseridae | Yes | | 44.6 |
| 3 | Mylopharyngodon piceus | Cyprinidae | Yes | | 57.2 |
| 4 | Corbicula fluminea | Cyrenidae | Yes | | 62.1 |
| 5 | Rana pipiens | Ranidae | | Yes | 96.4 |
| 6 | Daphnia pulicaria | Daphniidae | | Yes | 99 |
| 7 | Cyprinus carpio | Cyprinidae | Yes | | 106.3 |
| 8 | Eriocheir sinensis | Varunidae | Yes | | 112.9 |
| 9 | Ceriodaphnia dubia | Daphniidae | Yes | | 134.2 |
| 10 | Simocephalus vetulus | Daphniidae | Yes | | 142.9 |
| 11 | Acipenser brevirostrum | Acipenseridae | | Yes | 156.7 |
| 12 | Cyclops strenuus | Cyclopidae | Yes | | 162.2 |
| 13 | Physa gyrina | Physidae | | Yes | 164.5 |
| 14 | Daphnia magna | Daphniidae | Yes | | 164.8 |
| 15 | Tubifex | Tubificidae | | Yes | 216.5 |
| 16 | Cottus bairdii | Cottidae | | Yes | 222.2 |
| 17 | Limnodrilus hoffmeisteri | Tubificidae | Yes | | 241.5 |
| 18 | Anguilla | Anguillidae | | Yes | 286 |
| 19 | Macrobrachium nipponense | Palaemonidae | Yes | | 366.7 |
| 20 | Drunella grandis | Ephemerellidae | | Yes | 442.4 |
| 21 | Rana chensinensis | Ranidae | Yes | | 461.2 |
| 22 | Cipangopaludina cathayensis | Viviparidae | Yes | | 532.1 |
| 23 | Chironomus tentans | Chironomidae | Yes | | 546.2 |
| 24 | Misgurnus anguillicaudatus | Cobitidae | Yes | | 784.9 |
| 25 | Chironomus riparius | Chironomidae | Yes | | 1029 |

*Substitute species: the species are not distributed in Liaohe River Basin, but the species of the same family are distributed in Liaohe River Basin.

Step 3, supplementing toxicity values of site-specific resident sensitive species and endemic species;

After sorting the species in Table 1 according to the toxicity values, determining the four most sensitive families to ammonia nitrogen in Liaohe River Basin as: Cyprinidae, Acipenseridae, Cyrenidae and Ranidae. The process of determining sensitive species is as follows: for Cyprinidae, selecting Cyprinus carpio, as the species of Acipenseridae belong to protected animals, adding a test species, Pseudorasbora parva, to Cyprinidae; for cyrenidae, selecting Corbicula fluminea; as the species of Ranidae are amphibian and have poor laboratory availability, skipping to Daphniidae in the order of Table 1, and selecting Daphnia magna; and at this point, four sensitive test species are determined as Cyprinus carpio, Pseudorasbora parva, Corbicula fluminea and Daphnia magna.

According to Table 1, the most sensitive family of species in Liaohe River Basin is Cyprinidae, and according to the aquatic biota distribution characteristics of Liaohe River Basin, Abbottina liaoningensis and Ctenogobius giurinus are determined as endemic test species in Liaohe River

According to the step requirements of experiment guidelines in ASTM E1193-97 and ASTM E729-96, testing the above four sensitive test species and two endemic species for toxicity, and counting the number of deaths in each experimental group within 96 h for four species of tested fish; and counting the number of deaths in each experimental group within 48 h for two species of invertebrates, and calculating the median lethal concentration of the species based on the above experimental results.

With a species a as an example (in case of exposure to six different concentration series, each group is exposed to three parallel concentrations), calculating the toxicity value in the following process:

\textcircled{1} Counting the number of deaths of each experimental group of the species a under different exposure concentrations at the end of the toxicity test, and forming Table 2 through summarization;

TABLE 2
Summary of Deaths of Each Experimental Group of
Species a under Different Exposure Concentrations

| Exposure Concentration | Death | Initial Number |
|---|---|---|
| C1 | S1 | N |
| C1 | S2 | N |
| C1 | S3 | N |
| . . . | . . . | . . . |
| C6 | S16 | N |
| C6 | S17 | N |
| C6 | S18 | N |

\textcircled{2} Copying the three columns of data in Table 2 to SPSS software, selecting the three columns of data, clicking “Analysis”-“Regression”-“Probit”, selecting “Exposure Concentration” into the “Covariate” column, “Death” into the “Response Frequency” column and “Initial Number” into the “Summary of Observed Values” column, selecting “Log base is 10” in the “Transform” column, leaving the other options as the default, and clicking “OK”;

\textcircled{3} According to the SPSS output, determining the exposure concentration corresponding to a probability of 0.500 as the median lethal concentration LC50 of the species a.

According to the above process, determining the median lethal concentration LC50 of the six test species, that is the toxicity values of the six test species.

Step 4, establishing a resident toxicity database for the river basin;

Based on the supplementary toxicity values of six species and the existing toxicity database, replacing the toxicity values of Cyprinus carpio, Corbicula fluminea and Daphnia magna, and then supplementing the resident toxicity values of Pseudorasbora parva, Abbottina liaoningensis and Ctenogobius giurinus. As a result, the establishment of the resident toxicity database of ammonia nitrogen in Liaohe River Basin is completed, and all the species mean acute values are transformed to base-10 logarithmic values.

Step 5, comparing the degrees of fitting of species sensitivity distribution (SSD) models and identifying the optimal SSD model;

5.1) Calculating the cumulative probability of each species, wherein the calculation method is as follows: all the species are sorted in ascending order based on the species mean acute values, the serial number r of the species Leuciscuscephalus with the minimum toxicity value is 1, by analogy, the serial number of the species Chironomus riparius with the maximum toxicity value is 28, and the cumulative probability of the species is P=r/28+1, wherein 28 is the number of the species in sorting.

5.2) Transforming the species mean acute values of all the species to base-10 logarithms, wherein the final form is shown in Table 3.

TABLE 3
Resident Toxicity Database of Ammonia Nitrogen in Liaohe River Basin (all toxicity
values are based on the conditions of pH = 7.0 and temperature = 20° C.)

| | | | Species Mean | | |
|---|---|---|---|---|---|
| | | | Acute Value | Cumulative | Log₁₀ |
| Order | Species | Family | (SMAV) | Probability | (SMAV) |
| 1 | Leuciscus cephalus | Cyprinidae | 40.9 | 0.03 | 1.61 |
| 2 | Acipenser sinensis | Acipenseridae | 44.6 | 0.07 | 1.65 |
| 3 | Abbottina liaoningensis | Cyprinidae | 45.2 | 0.10 | 1.66 |
| 4 | Mylopharyngodon piceus | Cyprinidae | 57.2 | 0.14 | 1.76 |
| 5 | Ctenogobius giurinus | Gobiidae | 61.5 | 0.17 | 1.79 |
| 6 | Corbicula fluminea | Cyrenidae | 73.1 | 0.21 | 1.86 |
| 7 | Cyprinus carpio | Cyprinidae | 85.7 | 0.24 | 1.93 |
| 8 | Rana pipiens | Ranidae | 96.4 | 0.28 | 1.98 |
| 9 | Daphnia pulicaria | Daphniidae | 99 | 0.31 | 2.00 |
| 10 | Daphnia magna | Daphniidae | 100.1 | 0.34 | 2.00 |
| 11 | Eriocheir sinensis | Varunidae | 112.9 | 0.38 | 2.05 |
| 12 | Pseudorasbora parva | Cyprinidae | 116.2 | 0.41 | 2.07 |
| 13 | Ceriodaphnia dubia | Daphniidae | 134.2 | 0.45 | 2.13 |
| 14 | Simocephalus vetulus | Daphniidae | 142.9 | 0.48 | 2.16 |
| 15 | Acipenser brevirostrum | Acipenseridae | 156.7 | 0.52 | 2.20 |
| 16 | Cyclops strenuus | Cyclopidae | 162.2 | 0.55 | 2.21 |
| 17 | Physa gyrina | Physidae | 164.5 | 0.59 | 2.22 |
| 18 | Tubifex tubifex | Tubificidae | 216.5 | 0.62 | 2.34 |
| 19 | Cottus bairdii | Cottidae | 222.2 | 0.66 | 2.35 |
| 20 | Limnodrilus hoffmeisteri | Tubificidae | 241.5 | 0.69 | 2.38 |
| 21 | Anguilla anguilla | Anguillidae | 286 | 0.72 | 2.46 |
| 22 | Macrobrachium nipponense | Palaemonidae | 366.7 | 0.76 | 2.56 |
| 23 | Drunella grandis | Ephemerellidae | 442.4 | 0.79 | 2.65 |
| 24 | Rana chensinensis | Ranidae | 461.2 | 0.83 | 2.66 |
| 25 | Cipangopaludina cathayensis | Viviparidae | 532.1 | 0.86 | 2.73 |
| 26 | Chironomus tentans | Chironomidae | 546.2 | 0.90 | 2.74 |
| 27 | Misgurnus anguillicaudatus | Cobitidae | 784.9 | 0.93 | 2.89 |
| 28 | Chironomus riparius | Chironomidae | 1029 | 0.97 | 3.01 |

5.3) \textcircled{1} Copying the two columns of data including the cumulative probability of the species and the log-transformed toxicity value into Origin software, and selecting the cumulative probability of the species as “Y” and the log-transformed toxicity value as “X”;

\textcircled{2} Selecting the two columns of data “X” and “Y”, clicking “Plot”-“Symbol”-“Scatter”, then left clicking to select the generated dot plot, continuing to click “Analysis”-“Fitting”-“Nonlinear Curve Fit”, and using three function models normal, logistic and BurrIII for fitting, wherein the three functional relations are as follows:

The functional relation of the normal model is y=(1/(a*(2π)\hat{ }0.5))*exp((−(x−b)\hat{ }2)/(2*a\hat{ }2));

The functional relation of the logistic model is y=1/exp(−(x−a)/b);

The functional relation of the BurrIII model is y=1/(1+(1+a/x)\hat{ }b)\hat{ }c;

Using the above three functional relations for nonlinear fitting to obtain three different fit coefficients R².

\textcircled{3} Comparing the fit coefficients, and selecting the model with the best fitness as the optimal SSD model.

After nonlinear fitting, the normal distribution fit coefficient R² is 0.9315, the logistic distribution fit coefficient R² is 0.9887, and the BurIII distribution fit coefficient R² is 0.3265. The logistic model is determined as the optimal SSD model.

Step 6, validating the water quality criterion value.

\textcircled{1} Using the logistic model for nonlinear fitting of data, and based on the fitting curve (FIG. 2), using Y=0.05 to obtain X=1.4302;

\textcircled{2} Transforming the obtained X value to a base-10 exponent to obtain a value of 26.93 mg/L, and dividing this value by the safety coefficient 2 to obtain that the ammonia nitrogen water quality criterion value of Liaohe River Basin derived by the species sensitivity method is 13.47 mg/L.

\textcircled{3} Based on the toxicity percentage sorting method, using the toxicity values of four most sensitive species among the resident species and the sensitive species, wherein the four most sensitive species of resident sensitive species and endemic species in Liaohe River Basin are Abbottina liaoningensis, Ctenogobius giurinus, Corbicula fluminea and Cyprinus carpio, and based on the formula, obtaining that the ammonia nitrogen water quality criterion value of Liaohe River Basin derived by the toxicity sorting method is 15.91 mg/L.

\textcircled{4} The average cumulative probability of resident sensitive species and endemic species in Liaohe River Basin is 0.245 which is between 0 and 0.3, so the weight value obtained by the species sensitivity method is selected as 0.3, and the weight value obtained by the toxicity sorting method is selected as 0.7, thereby obtaining that the ammonia nitrogen water quality criterion value of Liaohe River Basin is 13.47×0.3+15.91×0.7=15.18 mg/L, which is the validation value for the ammonia nitrogen water quality criteria of Liaohe River Basin (under the conditions of pH=7.0 and temperature=20° C.), and the validation of the ammonia nitrogen water quality criteria of Liaohe River Basin is completed.

The above embodiments only express the implementation of the present invention, and shall not be interpreted as a limitation to the scope of the patent for the present invention. It should be noted that, for those skilled in the art, several variations and improvements can also be made without departing from the concept of the present invention, all of which belong to the protection scope of the present invention.

1. A method for validation of site-specific water quality criteria of a river basin, wherein the method comprises the following steps:
step 1: analyzing the biota distribution characteristics of a river basin;
step 2, eliminating non-resident related species from the existing toxicity values;
2.1) collecting, screening and summarizing the aquatic toxicity values of target pollutants;
2.2) comparing the resident species with the biological species having toxicity values in the river basin;
2.3) marking the species completely corresponding to the species names;
2.4) marking and reserving the species with different species names and the same species level based on biological classification as the reference;
2.5) eliminating the species that do not meet the requirements in steps 2.3) and 2.4);
step 3, supplementing toxicity values of site-specific resident sensitive species and endemic species;
3.1) screening the toxicity values of the species, wherein the screening principle is: the toxicity test subjects and the test process required to obtain toxicity values meet the requirements of the relevant toxicity test specifications, and after all qualified toxicity values of the species are screened, the species mean acute values SMAV of the finally reserved species in step 2 are calculated and sorted from small to large, and the calculation formula of the SMAV is as follows:
$$S M A V = \sqrt[n]{E {C}_{501} \times E {C}_{502} \times E {C}_{503} \times … \times {EC}_{50 n}}$$
wherein EC₅₀₁˜EC₅₀ₙ are the toxicity values of the same species, and n is the number of toxicity values of the same species; and EC50 can be substituted by median lethal concentration LC50;
3.2) selecting four species with the minimum SMAVs, and identifying four sensitive test species corresponding to four families according to the biological taxonomy of the species; in principle, each family corresponds to one species, but if no available test species exists in a family, amplifying one species from the higher sensitive family or identifying one substitute species from the families under sorting;
3.3) based on the family corresponding to the species with the minimum SMAV, identifying two endemic test species among the endemic species in the river basin;
3.4) conducting toxicity tests on the sensitive test species identified in step 3.2) and the endemic test species identified in step 3.3), and setting control tests for the toxicity tests, wherein the test species, exposure conditions and test procedures of the control group and the experimental group shall be exactly the same, the exposure concentration shall be set based on the principle of equal ratio, and the SPSS linear regression method is used to calculate EC50 or LC50 as the supplementary toxicity value;
step 4, establishing a resident toxicity database for the river basin;
4.1) if the sensitive test species already has a toxicity value, using the newly obtained toxicity value to replace the original toxicity value of the species;
4.2) adding the sensitive test species without previous toxicity values and the toxicity values of endemic test species to the original toxicity database;
4.3) according to the toxicity values, sorting all the species in ascending order to form the resident toxicity database of the river basin;
step 5, comparing the degrees of fitting of species sensitivity distribution SSD models and identifying the optimal SSD model;
5.1) calculating the cumulative probability P of the species;
5.2) taking all base-10 natural logarithmic values as toxicity values;
5.3) with the logarithm values of the toxicity values as independent variables and the cumulative probabilities of the species as dependent variables, respectively using normal, logistic and BurIII distribution models for fitting to obtain three different fit coefficients R²; and using the final fitting model derived with the maximum fit coefficient R² as the criterion as the optimum SSD model;
step 6, validating the water quality criterion value;
6.1) with the logarithm value of the toxicity value in the resident toxicity database of the river basin determined in step 4 as the X variable and the cumulative probability of the species as the Y variable, adopting the fitting models identified in step 5.3) for fitting;
6.2) taking the X value corresponding to Y=0.05 for base-10 exponent transformation and then dividing by the safety coefficient M to obtain that the site-specific water quality criterion value of the river basin of the target pollutant is 10^{X}/M;
6.3) based on the toxicity percentage sorting method, calculating the final toxicity value FV by using the toxicity values of four most sensitive species among the resident species and the sensitive species, and dividing the FV obtained by derivation by the safety coefficient to obtain the site-specific water quality criterion of the river basin, wherein the calculation formula of the final toxicity value is as follows:
$${S}^{2} = \frac{\sum [ {( ln SMAV )}^{2} ] - [ {( \sum ln SMAV )}^{2} / 4 ]}{\sum ( P ) - \frac{{[ \sum ( \sqrt{P} ) ]}^{2}}{4}} \\ L = \{ \sum ( ln SMAV ) - S [ \sum ( \sqrt{P} ) ] \} / 4 \\ A = S ( \sqrt{0.05} ) + L \\ F V = {e}^{A} \qquad$$
wherein S, L and A are respectively the parameters generated in the calculation process, SMAV is the species mean acute value, P is the cumulative probability corresponding to the species, and FV is the final toxicity value;
6.4) according to the resident sensitive species and the sorting of the toxicity of the sensitive species in the site-specific resident toxicity database, respectively selecting the weight values obtained by two different derivation methods;
TABLE 1

| | Ranges of Average Cumulative | | | |
|---|---|---|---|---|
| | Probabilities of Resident Sensitive | | | |
| | Species and Endemic Species | | | |
| | 0-0.30 | 0.31-0.50 | 0.51-0.80 | 0.81-1.0 |
| Weight value obtained based | 0.3 | 0.5 | 0.8 | 1.0 |
| on species sensitivity method | | | | |
| Weight value obtained based | 0.7 | 0.5 | 0.2 | 0.0 |
| on toxicity sorting method | | | | |

according to the weight values in the table above, obtaining the site-specific water quality criterion value of the river basin, as shown in the following formula:
WQC=WQCs×a+WQCr×b
wherein WQC is the final site-specific water quality criterion value of the river basin, WQCs is the site-specific water quality criterion value of the river basin derived by the species sensitivity method, WQCr is the site-specific water quality criterion value of the river basin derived by the toxicity sorting method, a is the weight value obtained based on the average cumulative probability of the resident sensitive species, and b is the weight value obtained based on the average cumulative probability of the endemic species.

2. The method for validation of site-specific water quality criteria of a river basin according to claim 1, wherein step 1 is specifically as follows:
1.1) collecting the fauna and local literature yearbooks of the river basin and summarizing species categories in the river basin;
1.2) sorting all site-specific species in order of genus, family and order from low to high according to biological taxonomy levels;
1.3) marking the endemic species according to the biological distribution characteristics of the river basin in combination with the data query of the species distribution area;
1.4) summarizing the biota distribution characteristics of the river basin.

3. The method for validation of site-specific water quality criteria of a river basin according to claim 1, wherein the ratio in the principle of equal ratio in step 3.4) is 2.

4. The method for validation of site-specific water quality criteria of a river basin according to claim 1, wherein the calculation method for the cumulative probability P of the species in step 5.1) is as follows: the species with the minimum toxicity value is assigned a value of 1, by analogy, the species with the maximum toxicity value is assigned a value of n, a total of n species are assumed, and the cumulative probability of the species is P=r/n+1, wherein n is the number of the species in sorting.

5. The method for validation of site-specific water quality criteria of a river basin according to claim 1, wherein the safety coefficient R in step 6.2) is 2.
